(19) United States
(12) Patent Application Publication
(10) Pub. No.: US 2025/0213784 A1
(43) Pub. Date: Jul. 3, 2025

(54) Systems and Methods for Enabling and Disabling Insulin Pumps in a Multi-Pump Insulin Pump System

(71) Applicant: Tandem Diabetes Care Switzerland Sàri, St-Sulpice (CH)

(72) Inventors: Pierre Fridez, Froideville (CH); Antoine Barraud, Lonay (CH); Virginia Lu, San Diego, CA (US); Patrick Segura, St-Sulpice (CH)

(21) Appl. No.: 19/005,328

(22) Filed: Dec. 30, 2024

Related U.S. Application Data
(60) Provisional application No. 63/616,479, filed on Dec. 29, 2023.

(51) Int. Cl. A61M 5/142

(52) U.S. Cl. CPC A61M 5/14236; A61M 5/14248; A61M 2205/52

(57) ABSTRACT

Systems and methods are provided for enabling and disabling insulin pumps in a multi-pump insulin pump system. For example, multiple pumps (e.g., patch pumps) may be rotated to deliver insulin to a user. In one example, one patch pump may be worn while another patch pump is charging. Once the battery of the patch pump worn by the user is depleted, a mobile device may cause the enabled pump in operation and worn by the user to send the latest pump settings and configuration data to the mobile device which will share the information with the disabled pump. The disabled pump will then update the pump settings based on this information and the mobile device may active the pump such that is now enabled for insulin delivery. The mobile device may also facilitate pairing between the patch pumps and a CGM device as they enabled for use and disabled.

### RELATED APPLICATIONS

This application claims priority to U.S. Provisional Patent App. No. 63/616,479 (filed Dec. 29, 2023), which is hereby incorporated in its entirety.

### TECHNICAL FIELD

This technology generally relates to the field of insulin pump systems. For example, systems and methods are provided herein for a multi-pump insulin system including more than one patch pump.

### BACKGROUND

Nearly 1 in 10 individuals in the United States are affected by diabetes. As technology advances, techniques for glucose monitoring and even insulin delivery so too develop and evolve. To manage the condition, historically many of these individuals were required to administer a blood draw, for example a needle prick in the fingertip to analyze the blood to determine blood glucose levels. If the blood glucose level did not satisfy a threshold, the individual may have to administer an insulin shot, meaning an injection of insulin into the subcutaneous tissue using a needle and syringe.

With advances in insulin pump and glucose monitoring technology, individuals may opt to use a wearable insulin pump which may be in communication with a continuous glucose monitor. Wearable insulin pumps selectively deliver a bolus based on preprogrammed settings for example. Such wearable insulin pumps often introduce insulin via an infusion site and are worn on the user's belt or sleeve or may even be adhered to the user as a patch pump. Patch pumps typically include a housing with all components including the pump, insulin cartridge, and battery, fully enclosed within a housing.

Wearable insulin pumps generally include a processor, memory, and/or storage for overseeing operation of the insulin pump based on programmed parameters as well as a battery. When the battery is depleted on a patch pump, the insulin pump portion of the patch pump must be removed from the user and the insulin pump portion connected to a charging device to charge the battery. During this time insulin will not be delivered to the user. While it may be desirable to use multiple pumps, it is logistically difficult to maintain the same insulin delivery settings (e.g., quantity, rate, intervals) as much of the insulin pump settings and configurations are saved locally on the pump. As a result, switching patch pumps may result in inconsistency in the delivery of insulin when the pumps are switched.

Accordingly, there is a need for systems and methods for enabling and disabling insulin delivery pumps without affecting insulin delivery and thus the quality of care provided to the user.

### SUMMARY

Provided herein are systems and methods for enabling and disabling insulin pumps in a multi-pump insulin pump system. Multiple pumps (e.g., patch pumps) may be used to deliver insulin to a user. One patch pump may be worn while another patch pump is charging. A mobile device may cause the pump worn by the user to be enabled such that the pump may deliver insulin to the user. The other pump may then be disabled and inoperable for delivering insulin. The mobile device may also facilitate pairing between the patch pumps as they are enabled for use and disabled.

Systems and method are provided herein for enabling and disabling insulin pumps in a multi-pump insulin pump system. The systems and methods may include memory designed to store computer-executable instructions and at least one computer processor designed to access the memory and execute the computer-executable instructions to determine to disable a first pump and enable a second pump in the multi-pump insulin pump system, request pump configuration data from the first pump, receive pump configuration data from the first pump, cause the second pump to adjust pump settings on the second pump based on the pump configuration data resulting in adjusted pump settings, cause the first pump to be disabled such that a first motor of the first pump is inoperable for pumping insulin, and cause the second pump to be enabled such that a second motor of the second pump is operable for pumping insulin based on the adjusted pump settings. The first pump and the second pump may each be patch pumps designed to be coupled to a pad adhered to a user, wherein each of the first pump and the second pump are configured to couple to an insulin cartridge including a volume of insulin and include a battery.

The system and method may further include requesting, prior to causing the second pump to be enabled, a battery charge amount from the second pump, receiving, the battery charge amount from the second pump, and determining the battery charge amount is above a threshold value corresponding to a minimum battery charge amount. Each first motor and second motor may be a screw motor. The system and method may further include requesting, prior to causing the second pump to be enabled, a screw position value from the second pump and corresponding to the second motor, receiving the screw position value from the second pump, and determining the screw position value is above a threshold value corresponding to a minimum screw position value.

The pump configuration data may include at least one of an insulin flow rate setting, historical insulin delivery data, historical continuous glucose monitoring data, or alarm settings. The first pump may be connected to a continuous glucose monitoring (CGM) device via a first wireless connection. The system and method may further include instructing, after causing the first pump to be disabled, the first pump to terminate the first wireless connection with the CGM device, determining a key code corresponding to the CGM device, sending the key code to the second pump, and causing the second pump to initiate a second wireless connection with the CGM device. The system and method may further include instructing, prior to instructing the first pump to terminate the first wireless connection, the first pump to send CGM data, receiving historical CGM data, and sending the historical CGM data to the second pump.

The system and method may include establishing a first wireless connection with the first pump, and establishing a second wireless connection with the second pump while maintaining the first wireless connection with the first pump, wherein the first wireless connection and the second wireless connection may be a short-range wireless connection. The system and method may include, prior to determining to disable the first pump and enable the second pump, determining a software version value corresponding the second pump, and determining the software version value satisfies a threshold value. The system and method may include sending, after the first pump is disabled but before the second pump is enabled, to the first pump and second pump a software update package, and causing the first pump and the second pump to execute the software update package.

The foregoing summary is illustrative only and is not intended to be in any way limiting. In addition to the illustrative aspects, embodiments, and features described above, further aspects, embodiments, and features will become apparent by reference to the following drawings and the detailed description.

### BRIEF DESCRIPTION OF THE DRAWINGS

FIG. 1 illustrates an exemplary multi-pump insulin pump system for enabling and disabling patch pumps in a multi-pump insulin system.

FIG. 2 illustrates an exemplary data flow depicting a mobile device connecting to multiple patch pumps and enabling and disabling the patch pumps.

FIG. 3 illustrates an exemplary process flow for disabling one patch pump and enabling a second patch pump in a multi-pump insulin pump system.

FIG. 4 illustrates an exemplary data flow depicting a mobile device connecting to multiple patch pumps and overseeing a connection between the patch pumps and a CGM device.

FIG. 5 illustrates an exemplary process flow for connecting one patch pump to a CGM device and then terminating the connection and connecting a second patch pump to the same CGM device.

FIG. 6 illustrates a schematic block diagram of a remote device in accordance with one or more exemplary embodiments of the disclosure.

The foregoing and other features of the present invention will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

The present disclosure is directed to systems and methods for enabling and disabling insulin pumps in a multi-pump insulin pump system. The multi-pump insulin pump system may include a mobile device and more than one insulin pump, which may each be a patch pump. The multi-pump insulin pump system may further include other devices such as a CGM device, a charging device, a server, a wearable device, and the like. A user may use the multi-pump insulin pump system to deliver insulin at certain times and/or for certain durations to regulate blood glucose levels for users with diabetes.

The patch pumps in the multi-pump insulin pump system may include a battery enclosed in a housing. To recharge the battery the patch pump may be disconnected from the user and connected to a charging device. While one pump of the multi-pump insulin pump system is charging, another pump of the multi-pump insulin pump system may be connected to the user such that the user receives insulin delivery with minimal interruption. Each pump may be connected to a mobile device and optionally to a CGM device.

As the pump in use and connected to the user is disconnected for charging or for any other reason (e.g., software update), the mobile device may oversee a transfer procedure to disable the pump in use and enable the new pump to be connected to the user. For example, certain pump settings, configuration data, operational data, and/or CGM data that is saved locally on the pump being removed from the patient may be sent to the mobile device and the mobile device may share this information with the new pump to be connected to the user. The mobile device may further cause the new pump to update pump settings and/or pump configuration based on the information and/or data received from the now removed pump. Additionally, the mobile device may oversee or otherwise facilitate pairing between each pump and the CGM device.

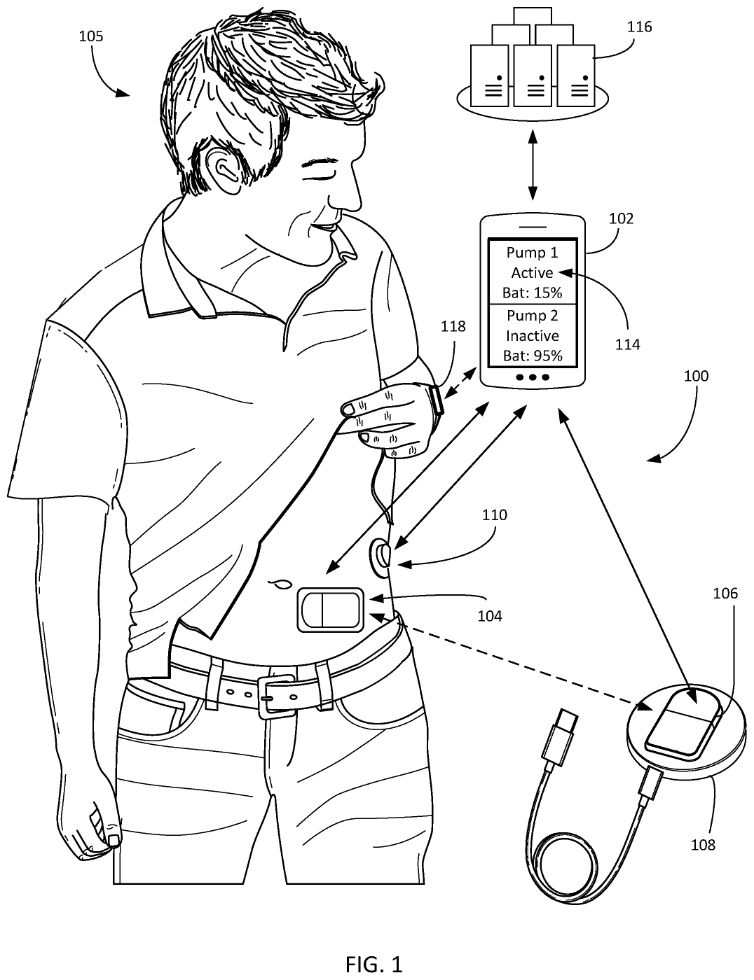

Referring now to FIG. 1, a multi-pump insulin pump system for enabling and disabling patch pumps in the multi-pump insulin system is depicted. Multi-pump system 100 may include patch pump 104, patch pump 106, and mobile device 102. Additionally, multi-pump system 100 may further include CGM device 110, server 116, charging device 108, wearable device 118 and/or one or more additional pumps, CGM devices, sensors, electronic devices, mobile devices and the like. While only two patch pumps are illustrated in FIG. 1, it is understood that more than two patch pumps may be used within multi-pump system 100. It is understood that wearable device 118 and/or server may alternatively perform one or more of the tasks and operations of mobile device 102 described herein.

Patch pump 104 and patch pump 106 may each be insulin pumps for pumping a volume of insulin according to pump parameters that set forth flow rates, duration, delivery sequence, and any suitable pump parameters. Patch pumps 104 and 106 may include certain hardware and software for selectively delivering a set amount of insulin to the user based on programmed settings, which may be tailored for a given user and adjusted over time. For example, operation of patch pumps 104 and 106 may be determined by software and/or settings loaded onto, saved onto, and preprogrammed on such pump. Similarly, mobile device 102 and/or server 116 may include certain software and/or settings for operating and/or adjusting patch pump 104 and 106.

Patch pumps 104 and 106 may each include a processor, memory, a communication unit, a battery (e.g., which may be a rechargeable battery and/or one or more batteries), an insulin container (e.g., insulin cartridge), a cannula for transcutaneous insulin delivery, and/or motor for pumping insulin into the user via the cannula, and/or any other suitable insulin pump components (e.g., visual indicators (e.g., lights), a screen, buttons, touchscreen, speaker, etc.).

Patch pumps 104 and 106 may be, for example, the same as or similar to the patch pump described in U.S. Patent No.______ issued on Dec. 20, 2022, and assigned to Tandem Diabetes Care Switzerland SÀRL, hereby incorporated by reference in its entirety. Patch pumps 104 and 106 may alternatively be wearable insulin pumps such as the insulin pump described in U.S. Patent App. Pub. No. 2014/0276423, published on Sep. 18, 2014 and assigned to Tandem Diabetes Care, Inc., and/or the insulin pump described in U.S. Patent App. Pub. No. 2011/014461, published on Jun. 16, 2011 and assigned to Tandem Diabetes Care, Inc., each of which are hereby incorporated by reference in their entirety.

Charging device 106 may connect to or otherwise electrically interface with patch pumps 104 and 106. For example, charging device 108 may electrically communicate with patch pumps 104 and 106 via inductive charging or a standard electrical connection. CGM device 110 may be any suitable continuous glucose monitoring sensor such as the Dexcom G7 CGM system available from Dexcom, Inc. of San Diego, California and/or other sensor device. Wearable device 118 may be any wearable device (e.g., smart watch, smart sensor, etc.) having a processor and memory and a communication unit for communicating with mobile device 102, patch pumps 104 and 106, CMG device 110 and/or server 116.

Mobile device 102 may be any suitable mobile device 102 (e.g., smart phone, smart device, mobile phone, tablet, etc.). Mobile device 102 may include a computer processor, memory, and/or a communication unit which may facilitate communication with any other devices (e.g., server 116, patch pumps 104 and 106 charging device 108). Mobile device 102 may optionally include a microphone, display (e.g., touchscreen), and/or any other input/output technology. Mobile device 102 may communicate with any devices in multi-pump insulin pump system 100 and any other devices via any suitable wireless system (e.g., Wi-Fi, cellular network, Bluetooth, Bluetooth Low Energy (BLE), near field communication protocol, etc.).

Remote server 116 may be any computing device having a processor, memory and a communication unit. For example, remote server Il 116 may be one or more servers, datastores, or the like. Remote device 11 16 may communicate with mobile device 1102 and/or any devices in insulin pump system 100 and any other devices via any well-known wired or wireless system (e.g., Wi-Fi, cellular network, Bluetooth, Bluetooth Low Energy (BLE), near field communication protocol, etc.).

As shown in FIG. 1, mobile device 102 may communicate with patch pumps 104 and 106 (e.g., via suitable wireless technology). Mobile device 102 may similarly communicate with CGM device 110 and smart device 118, as well as with server 116. In one example, server 116 may provide software updates for mobile device 102 and/or patch pumps 104 and 106. In one example, patch pump 104 may optionally communicate wirelessly with patch pump 106 (e.g., via any well-known wired or wireless system (e.g., Wi-Fi, cellular network, Bluetooth, Bluetooth Low Energy (BLE), near field communication protocol, etc.).

Mobile device 102 may be used by user 105 to enable and/or disable pump 104 and/or 106. For example, using mobile device 102, user 105 may view user interface 114 which may illustrate which insulin pumps that are available for pairing to mobile device and/or available for enabling and/or disabling for insulin delivery. User interface 114 may indicate that Pump 1, which may be pump 104, is active (e.g., enabled for insulin delivery) and has a battery level or charge of 15%, and Pump 2, which may be patch pump 106, is in active (e.g., disabled for insulin delivery) and has a battery level or charge of 95%. Based on this information, the user may determine to disconnect patch pump 104 to charge patch pump 104 and instead connect patch pump 106 to user 105 (e.g., via an adhesive pad).

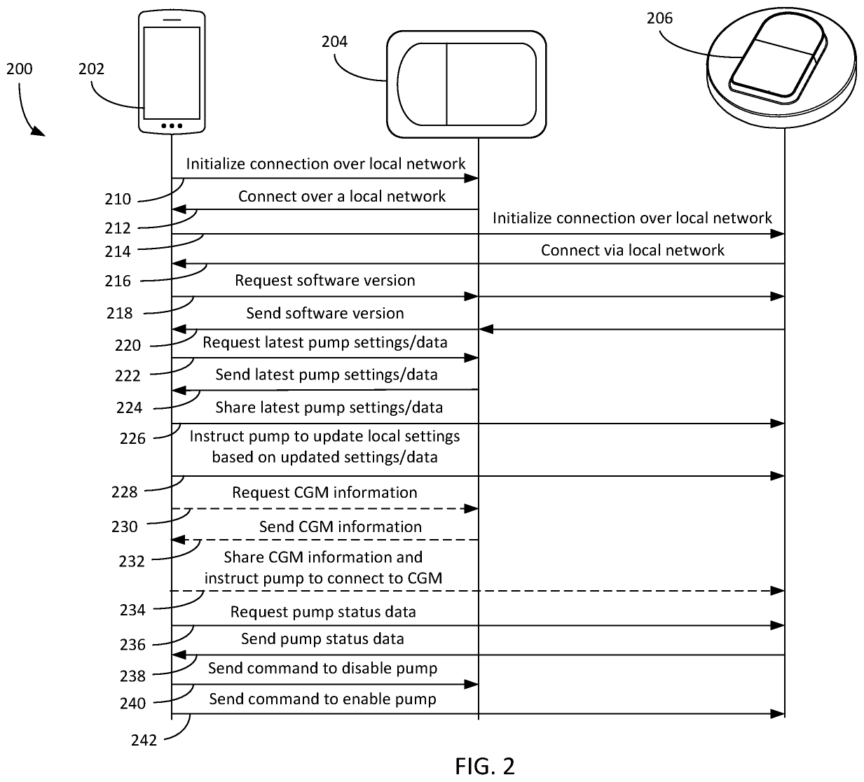

Referring now to FIG. 2, an exemplary data flow depicting a mobile device connecting to a multiple patch pumps and enabling and disabling the patch pumps is illustrated. Mobile device 202, pump 204, and pump 206 may be the same as or similar to mobile device 102, patch pump 204 and patch pump 206 of FIG. 1. Some or all of the operations of data flow 200 may be optional and may be performed in a different order.

To initiate data flow 200 to enable pump 204 and, at step 210, mobile device 202 may initialize a connection over a local network (e.g., Bluetooth) with pump 204 and at step 212 pump 204 may establish the connection with mobile device 202 and/or pair with mobile device 202. At step 214, mobile device 202 may initialize a connection over a local network (e.g., Bluetooth) with pump 206 and at step 216 pump 206 may establish the connection with mobile device 202 and/or pair with mobile device 202. In the example, illustrated in FIG. 2, mobile device 202 may connected to both pump 204 and 206 at the same time. The connection may be a short-range wireless connection, for example.

At step 218, mobile device 202 may request software version data from pumps 204 and/206 and at step 220, pumps 204 and/or 206 may share software version data indicating a software version number or type. Such information may be used by mobile device 202 to confirm that software running on the respective pump is compatible with software running on mobile device 202, and/or to confirm that pumps 204 and 206 are running the same software version.

At step 222, mobile device may request the latest pump setting data and/or pump configuration data from pump 204. In the example illustrated in FIG. 2, pump 204 may be initially enabled and operating on the user and pump 206 may be initially disabled. At step 224, pump 204 may send the latest pump setting data and/or pump configuration data to mobile device 202. For example, pump 204 may send user configuration profiles for insulin delivery, user settings for insulin delivery, historical insulin delivery data (e.g., flow rates, volumes, bolus information, durations, intervals, etc.), alarm settings, insulin flow rate settings, historical continuous glucose monitoring data, or and any other information saved locally on pump 204.

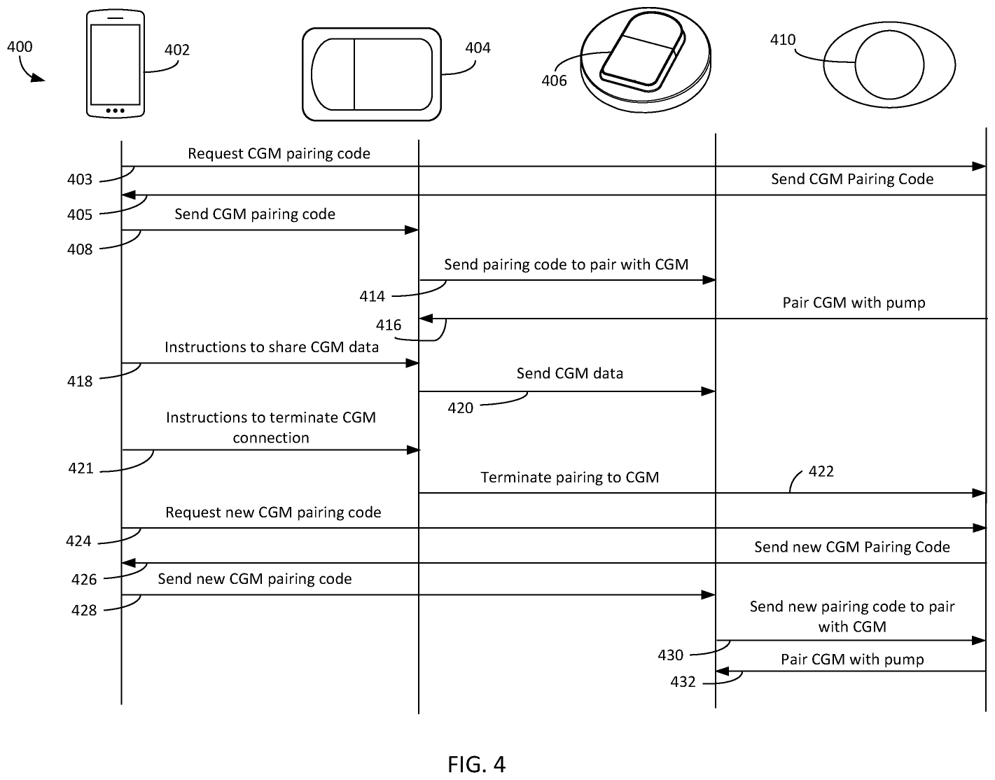

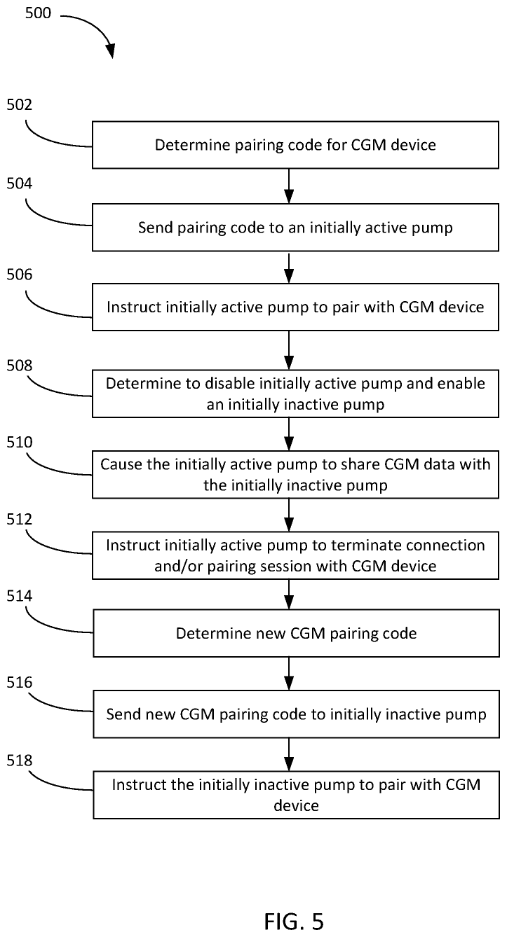

At step 226, mobile device 202 may share the latest pump settings, configuration data, or any other information received from pump 204 to pump 206 and at step 228 mobile device 202 may instruct the pump 206 to save the information sent and update local pump delivery settings on pump 206 based on this information. For example, insulin delivery settings and/or pump configurations may be adjusted on pump 206. At optional step 230, mobile device 202 may request CGM information from pump 204, at optional step 232 such CGM information and/or data may be sent from mobile device 202 to pump 206 and at optional step 234, such CGM information and/or data may be sent to pump 206 with instructions to connect to CGM. In one example, CGM information and/or data may be historical CGM data (e.g., blood glucose levels). The steps for connecting pump 206 to the CGM device are set forth in greater detail with respect to FIGS. 4 and 5.

At step 236, mobile device 202 may request pump status data from pump 206 and step 238, pump 206 may send pump status data to mobile device 202. Pump status data may include a battery charge value, a screw position for a screw motor, software version data if it has not already been sent to the mobile device, information regarding connection to adhesion pad (e.g., whether or not the pump is connected to the adhesion pad), information regarding cannula deployment, and the like. At step 240 mobile device 202 may send a command to disable pump 204 such that pump 204 is rendered inoperable for pumping insulin. At step 242, mobile device 202 may send a command to pump 206 to enable pump 206 such that pump 206 is operational and may deliver insulin.

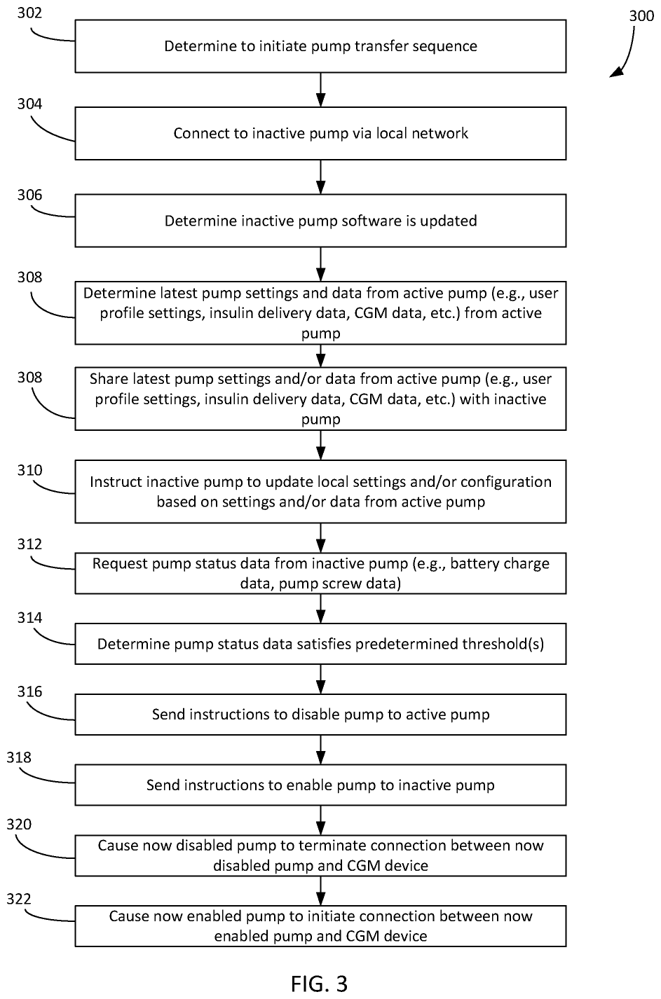

Referring now to FIG. 3, an exemplary process flow for disabling one patch pump and enabling a second patch pump in a multi-pump insulin pump system is illustrated. Process flow 300 may be performed by a mobile device (e.g., mobile device 102 of FIG. 1) and/or server (e.g., server 116 of FIG. 1). Some or all of the operations of process flow 200 may be optional and may be performed in a different order.

To initiate process flow 300, at block 302, computer-executable instructions stored on a memory of a device, such as a mobile device, may be executed to determine to initiate a pump transfer sequence. For example, a user may manually initiate the pump transfer on the user device or the mobile device and/or server may determine that a pump transfer is required (e.g., due to low battery of enabled pump attached to user, required software update, or any other reason). In one example, it may be desirable for the inactive pump to execute a software update via a software update package (e.g., sent to the mobile device by the server and distributed to the pump via the mobile device). In another example, it may be desirable to update both pumps when each pump is removed the user and/or disabled. For example, each pump may be disabled and caused to implement the software update package. At block 304, computer-executable instructions stored on a memory of a device, such as a mobile device, may be executed to connect the mobile device to an inoperable (e.g., inactive) pump via a local network (e.g., Bluetooth), if it is not already connected.

At block 306, computer-executable instructions stored on a memory of a device, such as a mobile device, may be executed to determine the inactive pump (e.g., the disabled pump) software is updated (e.g., the version running on the pump is the latest version) and/or compatible with the software running on the mobile device. At block 308, computer-executable instructions stored on a memory of a device, such as a mobile device, may be executed to determine the latest pump settings, configuration data, and/or other data from the active pump (e.g., the pump in operation and in use by the user). At block 308, computer-executable instructions stored on a memory of a device, such as a mobile device, may be executed to share the last pump settings, configuration data, and/or other data (e.g., user profile settings, insulin delivery data, CGM data, etc.) with the inactive pump.

At block 310, computer-executable instructions stored on a memory of a device, such as a mobile device, may be executed to instruct the inactive pump to update local pump delivery settings, configuration settings, and/or other pump settings based on the data or other information received at block 308. At block 312, computer-executable instructions stored on a memory of a device, such as a mobile device, may be executed to request pump status data from the inactive pump (e.g., the disabled pump). For example, battery charge data, pump screw data, and other data) may be requested. The pump may include a screw motor for dispensing insulin in an insulin cartridge and the screw data may include a position of the pump screw. In one example, the pump screw must be unwound to a certain position before the pump maybe be enabled again.

At block 314, computer-executable instructions stored on a memory of a device, such as a mobile device, may be executed to determine that the pump status data satisfies one or more predetermined thresholds. For example, the mobile device may determine whether the inactive pump has sufficient charge to be enabled (e.g., above 50% battery charge) and/or the pump screw entirely unscrewed (e.g., position is equal to 0% displacement or some other value). At block 316, computer-executable instructions stored on a memory of a device, such as a mobile device, may be executed to send instructions to the active pump to disable the active pump.

At block 318, computer-executable instructions stored on a memory of a device, such as a mobile device, may be executed to send instructions to the inactive pump to enable the inactive pump. At block 320, computer-executable instructions stored on a memory of a device, such as a mobile device, may be executed to cause the now disabled pump to terminate a connection and/or pairing of the CGM device. At block 322, computer-executable instructions stored on a memory of a device, such as a mobile device, may be executed to cause the now enabled device to initiate connection and/or pairing within the CGM device. Blocks 320 and 322 may be performed via the steps and operations described with respect to FIGS. 4 and 5.

Referring now to FIG. 4, an exemplary data flow depicting a mobile device connecting to a multiple patch pumps and overseeing connection between the patch pumps and a CGM device is depicted. Some or all of the operations of process flow 400 may be optional and may be performed in a different order. Mobile device 402, pump 404, pump 406, and CGM device 410 may be the same as or similar to mobile device 102, pump 104, pump 106, and/or CGM device 110 of FIG. 1.

To initiate process flow 400 to facilitate termination connection between one patch pump and a CGM device and initializing connection between another patch pump and the CGM device, at step 403, mobile device 402 may request a CGM pairing code from the CGM device. The pairing code may be any alphanumeric code used to pair an electronic device with the CGM device to establish a wireless connection. At step 405, the pairing device may send the CGM pairing code to the mobile device. Alternatively, a user may manually input the pairing code into the mobile device. At step 408, to pair pump 404) the initially enabled pump with the CGM device, the mobile device may send the CGM pairing code to the pump 404. At step 414 the pump 404 may initiate a connection with the CGM by sharing the pairing code with the CGM device. At block 416, the CGM device may accept or otherwise establish the connection with pump 404 (e.g., via a short-range network such as Bluetooth).

At step 418, mobile device 402 may instruct the pump 404 to share CGM data, which may include historical CGM data such as blood glucose levels from over a set period of time (e.g., the last five hours) and/or any other CGM data, with the disabled pump, pump 406. At step 420, the pump 44 may send such CGM to pump 406. At step 421, mobile device may send instructions to terminate the CGM connection and/or CGM pairing to pump 404. At step 422, the formerly active pump, pump 404, may terminate the short-range connection with the and/or paring with the CGM device.

At step 424, mobile device may request a new pairing code for a connection over a short-range network from the CGM device. At step 426, the CGM device may send a new 12 pairing code to mobile device 402 and at step 428 the new pairing code may be sent from the mobile device to pump 406, which may be enabled for pumping insulin and thus activated. At step 430, pump 406 may send the new pairing code to the CGM device to initiate a local connection with the CGM device. At step 432, the CGM device may accept or otherwise establish the short-range (e.g., Bluetooth) connection and/or pairing.

Referring now to FIG. 5, an exemplary process flow for connecting one patch pump to a CGM device and then terminating the connection and connecting a second patch pump to the same CGM device is depicted. Process flow 500 may be performed by a mobile device (e.g., mobile device 102 of FIG. 1). Some or all of the operations of process flow 500 may be optional and may be performed in a different order.

To initiate process flow 500, at block 502, computer-executable instructions stored on a memory of a device, such as a mobile device, may be executed to determine a pairing code to connect a pump to a CGM device. For example, the pairing code may be an alphanumeric code or any other value or key. The pairing code may be manually input by a user, may be received from the CGM device, or may otherwise be determined. At block 504, computer-executable instructions stored on a memory of a device, such as a mobile device, may be executed to send the paring code to the initially active pump (e.g., a pump that is enabled and connected to the user).

At block 506, computer-executable instructions stored on a memory of a device, such as a mobile device, may be executed to instruct the initially active pump to pair with the CGM device via the pairing code (e.g., to pair with the CGM over a short-range network). At block 508, computer-executable instructions stored on a memory of a device, such as a mobile device, may be executed to disable an initially active pump and enable an initially inactive pump. At block 510, computer-executable instructions stored on a memory of a device, such as a mobile device, may be executed to cause the initially active pump to share CGM data with the initially inactive pump. The CGM data may be historic CGM data over a certain period of time (e.g., the last five hours of CGM data).

At block 512, computer-executable instructions stored on a memory of a device, such as a mobile device, may be executed to instruct the initially active pump to terminate the connection and/or pairing session (e.g., wireless connection) with the CGM device. At block 512, computer-executable instructions stored on a memory of a device, such as a mobile device, may be executed to determine new CGM pairing code from the CGM device. At block 516, computer-executable instructions stored on a memory of a device, such as a mobile device, may be executed to send the new CGM pairing code to the initially inactive pump. At block 518, computer-executable instructions stored on a memory of a device, such as a mobile device, may be executed to instruct the initially inactive pump to pair with the CGM device (e.g., using the pairing code).

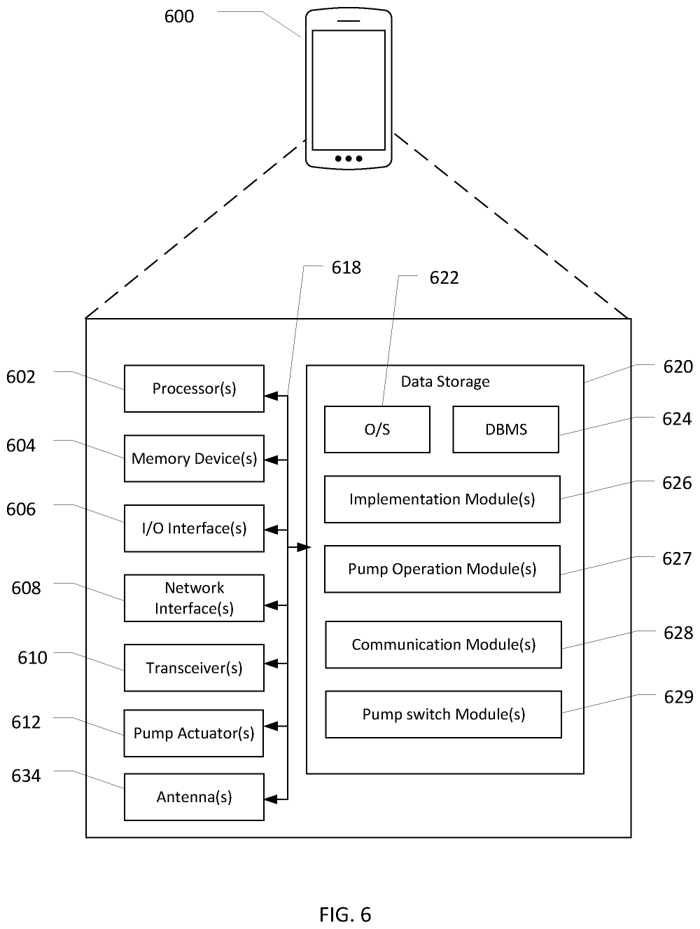

Referring now to FIG. 6 a schematic block diagram of illustrative mobile device 600, which may be in communication with one or more insulin pumps (e.g., patch pumps), servers, and/or wearable devices is illustrated. Mobile device 600 may be the same or similar to mobile device 102 of FIG. 1 or otherwise one or more of the mobile device of FIGS. 2 to 5. It is understood that mobile device 600 may alone or together with one or more devices of the multi-pump insulin pump system perform one or more of the operations of mobile device 600.

Mobile device 600 may be designed to communicate with one or more pumps, remote servers, wearables, charging devices, other systems, or the like. Mobile device 600 may be designed to communicate via one or more networks. Such network(s) may include, but are not limited to, any one or more different types of communications networks such as, for example, near field communication networks, cable networks, public networks (e.g., the Internet), private networks (e.g., frame-relay networks), wireless networks, cellular networks, telephone networks (e.g., a public switched telephone network), or any other suitable private or public packet-switched or circuit-switched networks.

In an illustrative configuration, mobile device 600 may include one or more processors 602, one or more memory devices 604 (also referred to herein as memory 604), one or more input/output (I/O) interface(s) 606, one or more network interface(s) 608, one or more transceiver(s) 610, one or more pump actuator(s) 612, one or more antenna(s) 634, and data storage 620. Mobile device 600 may further include one or more bus(es) 618 that functionally couple various components of the mobile device 600.

The bus(es) 618 may include at least one of a system bus, a memory bus, an address bus, or a message bus, and may permit exchange of information (e.g., data (including computer-executable code), signaling, etc.) between various components of the mobile device 600. The bus(es) 618 may include, without limitation, a memory bus or a memory controller, a peripheral bus, an accelerated graphics port, and so forth. The bus(es) 618 may be associated with any suitable bus architecture including.

The memory 604 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth.

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein. The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

The data storage 620 may store one or more operating systems (O/S) 622; one or more optional database management systems (DBMS) 624; and one or more program module(s), applications, engines, computer-executable code, scripts, or the like such as, for example, one or more implementation modules 626, one or more pump operation modules 627, one or more communication modules 628, and/or one or more pump switch modules 629. Some or all of these modules may be sub-modules. Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

Referring now to other illustrative components depicted as being stored in data storage 620, O/S 622 may be loaded from data storage 620 into memory 604 and may provide an interface between other application software executing on mobile device 600 and hardware resources of the mobile device 600. More specifically, O/S 622 may include a set of computer-executable instructions for managing hardware resources of mobile device 600 and for providing common services to other application programs (e.g., managing memory allocation among various application programs). In certain example embodiments, O/S 622 may control execution of the other program module(s) for content rendering. O/S 622 may include any operating system now known or which may be developed in the future including, but not limited to, any server operating system, any mainframe operating system, or any other proprietary or non-proprietary operating system.

Optional DBMS 624 may be loaded into the memory 604 and may support functionality for accessing, retrieving, storing, and/or manipulating data stored in memory 604 and/or data stored in data storage 620. DBMS 624 may use any of a variety of database models (e.g., relational model, object model, etc.) and may support any of a variety of query languages. DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like.

I/O interface(s) 606 may facilitate the receipt of input information by mobile device 600 from one or more I/O devices as well as the output of information from mobile device 600 to the one or more I/O devices. The I/O devices may include any of a variety of components such as a touchscreen display; an audio output device for producing sound, such as a speaker; an audio capture device, such as a microphone; buttons and/or dials; and so forth. Any of these components may be integrated into mobile device 600 or may be separate.

Mobile device 600 may further include one or more network interface(s) 608 via which mobile device 600 may communicate with any of a variety of other systems, platforms, networks, devices, and so forth. Network interface(s) 608 may enable communication, for example, with one or more wireless routers, one or more host servers, one or more web servers, and the like via one or more of networks.

Antenna(s) 634 may include any suitable type of antenna depending, for example, on the communications protocols used to transmit or receive signals via antenna(s) 634. Nonlimiting examples of suitable antennas may include directional antennas, non-directional antennas, dipole antennas, folded dipole antennas, patch antennas, multiple-input multiple-output (MIMO) antennas, or the like. Antenna(s) 634 may be communicatively coupled to one or more transceivers 610 or radio components to which or from which signals may be transmitted or received. Antenna(s) 634 may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals including BLE signals, a Near Field Communication (NFC) antenna for transmitting or receiving NFC signals, a 900 MHz antenna, and so forth.

Transceiver(s) 610 may include any suitable radio component(s) for, in cooperation with the antenna(s) 634, transmitting or receiving radio frequency (RF) signals in the bandwidth and/or channels corresponding to the communications protocols utilized by mobile device 600 to communicate with other devices. Transceiver(s) 610 may include hardware, software, and/or firmware for modulating, transmitting, or receiving potentially in cooperation with any of antenna(s) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. Transceiver(s) 610 may further include hardware, firmware, or software for receiving GNSS signals. Transceiver(s) 610 may include any known receiver and baseband suitable for communicating via the communications protocols utilized by the mobile device 600. The transceiver(s) 610 may further include a low noise amplifier (LNA), additional signal amplifiers, an analog-to-digital (A/D) converter, one or more buffers, a digital baseband, or the like.

Referring now to functionality supported by the various program module(s) depicted in FIG. 6, implementation module(s) 626 may include computer-executable instructions, code, or the like that responsive to execution by one or more of the processor(s) 602 may perform functions including, but not limited to, overseeing coordination and interaction between one or more modules and computer executable instructions in data storage 620, determining user actions, determining actions associated with user interactions, determining actions associated with user input, initiating commands locally or at periphery and/or remote devices, and the like.

Pump operation module(s) 627 may include computer-executable instructions, code, or the like that responsive to execution by one or more of the processor(s) 602 may perform functions including, but not limited to, overseeing, monitoring, and/or causing the pump to operate to deliver a select volume of insulin.

Communication module(s) 628 may include computer-executable instructions, code, or the like that responsive to execution by one or more of the processor(s) 602 may perform functions including, but not limited to, communicating with one or more devices, for example, via wired or wireless communication, communicating with mobile devices, smart devices, remote devices, charging devices, computing devices, smart sensors and/or any other devices.

The pump switch module(s) 629 may include computer-executable instructions, code, or the like that responsive to execution by one or more of the processor(s) 602 may perform functions including, but not limited to, overseeing and facilitating selectively enabling and disabling insulin pumps in the multi-pump insulin pump system and overseeing connection to a CGM.

Although specific embodiments of the disclosure have been described, one of ordinary skill in the art will recognize that numerous other modifications and alternative embodiments are within the scope of the disclosure. For example, any of the functionality and/or processing capabilities described with respect to a particular device or component may be performed by any other device or component. Further, while various illustrative implementations and architectures have been described in accordance with embodiments of the disclosure, one of ordinary skill in the art will appreciate that numerous other modifications to the illustrative implementations and architectures described herein are also within the scope of this disclosure.

Certain aspects of the disclosure are described above with reference to block and flow diagrams of systems, methods, apparatuses, and/or computer program products according to example embodiments. It will be understood that one or more blocks of the block diagrams and flow diagrams, and combinations of blocks in the block diagrams and the flow diagrams, respectively, may be implemented by execution of computer-executable program instructions. Likewise, some blocks of the block diagrams and flow diagrams may not necessarily need to be performed in the order presented, or may not necessarily need to be performed at all, according to some embodiments. Further, additional components and/or operations beyond those depicted in blocks of the block and/or flow diagrams may be present in certain embodiments.

Accordingly, blocks of the block diagrams and flow diagrams support combinations of means for performing the specified functions, combinations of elements or steps for performing the specified functions, and program instruction means for performing the specified functions. It will also be understood that each block of the block diagrams and flow diagrams, and combinations of blocks in the block diagrams and flow diagrams, may be implemented by special-purpose, hardware-based computer systems that perform the specified functions, elements or steps, or combinations of special-purpose hardware and computer instructions.

Program module(s), applications, or the like disclosed herein may include one or more software components, including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component including assembly language instructions may require conversion into executable machine code by an assembler prior to execution by the hardware architecture and/or platform.

Another example programming language may be a higher-level programming language that may be portable across multiple architectures. A software component including higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component including instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may include other custom-developed application software, operating system functionality (e.g., device drivers, data storage (e.g., file management) routines, other common routines, and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a CRSM that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or arc to be performed in any particular embodiment.

It should be understood that any of the computer operations described herein above may be implemented at least in part as computer-readable instructions stored on a computer-readable memory. It will of course be understood that the embodiments described herein are illustrative, and components may be arranged, substituted, combined, and designed in a wide variety of different configurations, all of which are contemplated and fall within the scope of this disclosure.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A method for enabling and disabling insulin pumps in a multi-pump insulin pump system, the method comprising:
determining to disable a first pump and enable a second pump in the multi-pump insulin pump system;
requesting pump configuration data from the first pump;
receiving pump configuration data from the first pump;
causing the second pump to adjust pump settings on the second pump based on the pump configuration data resulting in adjusted pump settings;
causing the first pump to be disabled such that a first motor of the first pump is inoperable for pumping insulin; and
causing the second pump to be enabled such that a second motor of the second pump is operable for pumping insulin based on the adjusted pump settings.

2. The method of claim 1, wherein the first pump and the second pump are each patch pumps configured to be coupled to a pad adhered to a user, wherein each of the first pump and the second pump are configured to couple to an insulin cartridge comprising a volume of insulin and comprise a battery.

3. The method of claim 2, further comprising:
requesting, prior to causing the second pump to be enabled, a battery charge amount from the second pump;
receiving, the battery charge amount from the second pump; and
determining the battery charge amount is above a threshold value.

4. The method of claim 2, wherein each first motor and second motor is a screw motor, the method further comprising:
requesting, prior to causing the second pump to be enabled, a screw position value from the second pump and corresponding to the second motor;
receiving the screw position value from the second pump; and
determining the screw position value is above a threshold value.

5. The method of claim 1, wherein the pump configuration data comprises at least one of an insulin flow rate setting, historical insulin delivery data, historical continuous glucose monitoring data, or alarm settings.

6. The method of claim 1, wherein the first pump is connected to a CGM device via a first wireless connection, the method further comprising:
instructing, after causing the first pump to be disabled, the first pump to terminate the first wireless connection with the CGM device;
determining a key code corresponding to the CGM device;
sending the key code to the second pump; and
causing the second pump to initiate a second wireless connection with the CGM device.

7. The method of claim 6, further comprising:
instructing, prior to instructing the first pump to terminate the first wireless connection, the first pump to send CGM data;
receiving historical CGM data; and
sending the historical CGM data to the second pump.

8. The method of claim 1, further comprising:
establishing a first wireless connection with the first pump; and
establishing a second wireless connection with the second pump while maintaining the first wireless connection with the first pump;
wherein the first wireless connection and the second wireless connection are shortrange wireless connections.

9. The method of claim 1, further comprising:
prior to determining to disable the first pump and enable the second pump, determining a software version value corresponding the second pump; and
determining the software version value satisfies a threshold value.

10. The method of claim 1, further comprising:
sending, after the first pump is disabled but before the second pump is enabled, the first pump and second pump a software update package; and
causing the first pump and the second pump to execute the software update package.

11. A device comprising:
memory configured to store computer-executable instructions; and
at least one computer processor configured to access the memory and execute the computer-executable instructions to:
determine to disable a first pump and enable a second pump in a multi-pump insulin pump system;
request pump configuration data from the first pump;
receive pump configuration data from the first pump;
cause the second pump to adjust pump settings on the second pump based on the pump configuration data resulting in adjusted pump settings; cause the first pump to be disabled such that a first motor of the first pump is inoperable for pumping insulin; and
cause the second pump to be enabled such that a second motor of the second pump is operable for pumping insulin based on the adjusted pump settings.

12. The device of claim 11, wherein the first pump and the second pump are each patch pumps configured to be coupled to a pad adhered to a user; wherein each of the first pump and the second pump are configured to couple to an insulin cartridge comprising a volume of insulin and comprise a battery.

13. The device of claim 12, wherein the at least one computer processor is further configured to access the memory and execute the computer-executable instructions to:
request, prior to causing the second pump to be enabled, a battery charge amount from the second pump;
receive, the battery charge amount from the second pump; and
determine the battery charge amount is above a threshold value.

14. The device of claim 12, wherein each first motor and second motor is a screw motor and the at least one computer processor is further configured to access the memory and execute the computer-executable instructions to:
request, prior to causing the second pump to be enabled, a screw position value from the second pump and corresponding to the second motor;
receive the screw position value from the second pump; and
determine the screw position value is above a threshold value.

15. The device of claim 11, wherein the pump configuration data comprises at least one of an insulin flow rate setting, historical insulin delivery data, historical continuous glucose monitoring data, or alarm settings.

16. The device of claim 11, wherein the first pump is connected to a CGM device via a first wireless connection and wherein the at least one computer processor is further configured to access the memory and execute the computer-executable instructions to:
instruct, after causing the first pump to be disabled, the first pump to terminate the first wireless connection with the CGM device;
determine a key code corresponding to the CGM device;
send the key code to the second pump; and
cause the second pump to initiate a second wireless connection with the CGM device.

17. The device of claim 16, wherein the at least one computer processor is further configured to access the memory and execute the computer-executable instructions to:
instruct, prior to instructing the first pump to terminate the first wireless connection, the first pump to send CGM data;
receive historical CGM data; and
send the historical CGM data to the second pump.

18. The device of claim 17, the at least one computer processor is further configured to access the memory and execute the computer-executable instructions to:
establish a first wireless connection with the first pump; and
establish a second wireless connection with the second pump while maintaining the first wireless connection with the first pump;
wherein the first wireless connection and the second wireless connection are shortrange wireless connections.

19. The device of claim 11, wherein the at least one computer processor is further configured to access the memory and execute the computer-executable instructions to:
prior to determining to disable the first pump and enable the second pump, determine a software version value corresponding the second pump; and
determine the software version value satisfies a threshold value.

20. The device of claim 11, wherein the at least one computer processor is further configured to access the memory and execute the computer-executable instructions to:
send, after the first pump is disabled but before the second pump is enabled, the first pump and second pump a software update package; and
cause the first pump and the second pump to execute the software update package.
